(19) United States
(12) Patent Application Publication
(10) Pub. No.: US 2003/0027347 A1
(43) Pub. Date: Feb. 6, 2003

(54) Automated method for correcting blood analysis parameter results affected by interference from exogenous blood substitutes in whole blood, plasma, and serum

(76) Inventor: Phyllis Shapiro, Stamford, CT (US)

Correspondence Address: MORGAN & FINNEGAN, L.L.P., 345 Park Avenue, New York, NY, 10154-0053, US

(21) Appl. No.: 09/865,759

(22) Filed: May 25, 2001

(51) Int. Cl. G01N 33/72; G06F 19/00; G01N 33/48; G01N 33/50

(52) U.S. Cl. 436/66; 702/19

(57) ABSTRACT

The invention describes an automated method for correcting interferences with blood chemistry results on plasma or serum using automated hematology analysis of a whole blood sample. Such interference error results from the presence of exogenous oxygen-carrying blood substitutes in transfused blood samples. The automated method is performed using automated hematology analysis to correct errors due to interference in the determination of blood chemistries to provide accurate quantification of these parameters directly, rapidly and automatically. The automated interference correction method is advantageous for medical and clinical use following transfusion of patients with blood substitutes after trauma or during surgery, and for repeated or periodic monitoring of patients' blood samples during recovery. The inventive method can also be used to correct for any in vivo hemolysis, or in-collection-tube hemolysis if both the chemistry results and the cell by cell measurements are performed on blood from the same collection tube.

### FIELD OF THE INVENTION

The present invention relates generally to new methods for correcting interference to hematology and clinical chemistry parameters. Interference can occur during the analysis of whole blood, plasma and serum samples due to the presence of cell-free blood substitutes which are added to a patient's blood as supplementary oxygen carriers.

### BACKGROUND OF THE INVENTION

Whole blood substitutes have long been sought after as alternatives to whole blood for use in the medical field, particularly following trauma and/or surgery where transfusions are needed. Currently, there is a renewed interest to produce and/or isolate one or more blood substitutes. However, because of the complexity of blood and the various components that comprise whole blood, as well as the stringent federal regulations governing the testing and use of such synthetic products, industry has focused its research efforts on the development of products which temporarily deliver oxygen, rather than on the development of a variety of different products having other functions that transfused blood provides.

Hemoglobin (HGB) isolated from human or animal (e.g., bovine) blood, or a synthetically produced oxygen carrier, such as perfluorocarbon (PFC), are two types of hemoglobin substitutes that are currently in clinical trials. Other red blood cell substitutes, i.e., oxygen-carrying hemoglobin substitutes, have also been developed and characterized for use in patients. (See, for example, Red Blood Cell Substitutes, 1998, (Eds.) A. S. Rudolph, R. Rabinovici, and G. Z. Feuerstein, Dekker, New York, N.Y.). Such oxygen-carrying hemoglobin substitutes may be used in conjunction with standard medical therapies, such as transfused blood or blood products. Indeed, the interest in the use of temporary oxygen carriers as blood substitutes is expected to increase as a means of reducing requirements for allogeneic blood. (Z. Ma et al., 1997, Clin. Chem., 43:1732-1737).

As a specific but nonlimiting example, Enzon, Inc. (Piscataway, N.J.), has developed a polyethylene glycol (PEG)-modified bovine hemoglobin, abbreviated PEG-HGB. PEG-HGB is produced by a process in which strands of PEG are crosslinked to the surfaces of HGB molecules, for example, as disclosed in U.S. Pat. Nos. 5,386,014 and 5,234,903 to Nho et al.).

The first generation HGB substitutes were generally intended for short term treatment of blood/oxygen loss during surgery or following trauma. One disadvantage of HGB substitutes is the short circulation half-life attributed to these products. For example, HGB substitutes that are added to blood have a circulation half-life of up to 36 hours compared with a circulation half-life of up to 30 days for transfused blood. However, this relatively short half-life is typically not a serious problem associated with the use of such blood substitutes, because these substitutes are predominantly indicated for short-term treatment objectives.

In general, the measurement of hemoglobin in whole blood samples is performed by commercially available hematology analyzers. To date, with the exception of certain automated hematology analyzers, such as those available from Bayer Corporation, e.g., the ADVIA 120® automated hematology analyzer system, other commercially-available blood analyzers are able to measure only total hemoglobin, which includes not only exogenously added hemoglobin, but also intracellular hemoglobin that is derived from the red blood cells in a blood sample.

Patent application U.S. Ser. No. 60/210,625, filed Jun. 9, 2000, describes automated methods for determining and measuring exogenous hemoglobin in a whole blood sample in a reliable and reproducible way. The automated methods described therein provide the ability to monitor, repeatedly or periodically during a course or regimen of a patient's treatment, heme-colored hemoglobin, and/or a hemoglobin product, derivative or substitute, such as a cell-free hemoglobin derivative, that has been added to blood of a patient. Also described are methods for monitoring, determining, or quantifying a hemoglobin product, or a substance containing the product (e.g., blood, plasma, or a physiologically acceptable solution or composition, and the like) after transfusing a patient with such a hemoglobin product, derivative or substitute, such as a cell-free hemoglobin derivative. The disclosure further provides a system to differentiate and accurately measure the contribution of an added or exogenous hemoglobin product or blood substitute, e.g., PEG-HGB, separately and distinctly from the contribution of cellular HGB which derives from a patient's red blood cells. The automated analytical method and system as described calculate a specific concentration of the extracellular hemoglobin in a blood sample to which a hemoglobin product has been added, or in a sample which contains extracellular hemoglobin to be detected to allow the detection and monitoring of an extracellular hemoglobin component, even in the presence of a cellular hemoglobin component derived from the red blood cells in a given sample.

Clinical laboratory assays play an important role in the care of many perioperative or postoperative patients and trauma victims. Such assays are also required to monitor and care for patients who receive blood substitutes. Both hemolysis and lipemia are known to cause interference in many calorimetric and spectrophotometric methods used in clinical laboratories (O. Sonntag, 1986, J. Clin. Chem. Biochem., 24:127-139; W. G. Guder, 1986, J. Clin. Chem. Biochem., 24:125-126; and J. P. Chapelle et al., 1990, Clin. Chem., 36:99-101). Hemolysis causes interference because of the strong optical absorbances of heme-colored hemoglobin species between 500 and 600 nm, while lipemia causes colorless interference because of light scattering. For example, after bovine hemoglobin-based oxygen carrying (HBOC) solution is administered to patients, there is a dose-related presence of soluble hemoglobin in plasma and a marked red coloration of plasma. In these patients, plasma hemoglobin values can be as great as 50 g/L, which is well above the concentrations of hemoglobin described as interfering in many laboratory assays (see supra).

In addition, perfluorocarbon emulsion dosing concentrations of 3.0-4.5 mL/kg result in a dilution of approximately 1:20-1:25 of the perfluorocarbon in blood, such that plasma samples from these patients can have a lipemic appearance. Neither lipemia nor the effects of perfluorocarbon emulsions is addressed by this invention.

In view of the above, it is important for clinical laboratories to determine which tests and test results are valid when performed on samples from patients receiving these and other blood substitutes.

The most common preanalytic factor affecting the acceptability of specimens or samples (e.g., blood samples) for analysis is the presence of interfering substances within the specimen or sample. The presence of interfering substances, e.g., exogenously added hemoglobin derivatives and oxygen-carrying blood substitutes, alters the correct value of a measured result and may lead to inappropriate clinical intervention and compromise patient outcome. (S. C. Kazmierczak and P. G. Catrou, 2000, “Analytical interference. More than just a laboratory problem”, Am. J. Clin. Pathol., 113(1):9-11).

The interference of exogenous hemoglobin, or oxygen-carrying blood substitutes, in a blood sample with the measurement of mean cell hemoglobin (MHC) value, mean cell hemoglobin concentration (MCHC) value, as well as with a number of blood chemistry assays can be corrected by a manual (unautomated) multistep process which requires centrifuging an anticoagulated whole blood sample and obtaining a measurement of the plasma hemoglobin. The plasma hemoglobin (or serum hemoglobin) measurement is then used to recalculate manually the erroneous results. For example, for hematology:

Red Blood Cell Hemoglobin, or Cell Hemoglobin, (RBC HGB or Cell HGB), [units: gm/dL]=Total HGB−Plasma HGB [units: gm/dl]

MCH, (corrected), [units: picograms/cell]=RBC HGB/RBC count [count units: cells/mm³] (×10)

MCHC [units: gm/dl]=RBC HGB/Hematocrit (HCT) [%] (×100);

and for chemistry:

Corrected Result=Reported Result−(Correction Factor×Serum Hemoglobin or Plasma Hemoglobin [units: (gm/dL)].

Correction factors are routinely and empirically determined by individual clinical laboratories for various blood parameters. In the above equations and in equations in which these parameters are specified hereinbelow, the units for Red Blood Cell Hemoglobin (RBC HGB), (also called Cell Hemoglobin), are gm/dL; the units for Plasma HGB are gm/L; the units for MCH are picograms/cell; the units for RBC concentration, or cell count, are cells/mm³; the units for MCHC are gm/dL; and the unit for Hematocrit (HCT) is %.

The present invention provides a solution to a problem which accompanies the use of exogenous blood substitutes, e.g., hemoglobin substitutes, or oxygen-carrying blood substitutes, and the analysis of blood, plasma and serum samples by hematology analyzers. The problem is that of interference, e.g., interference caused by cell free hemoglobin derivative, or blood substitute, compounds, which have a color to them, and/or other oxygen-carrying blood substitutes, in patient samples. These substances interfere with certain clinical chemistry and hematology test values and result parameters.

The present invention solves this problem by the discovery and provision of an automated method to correct blood analysis parameter results to account for interference error. Accordingly, the present invention provides a faster, less time consuming, fully-automated way to obtain accurate results of clinical chemistries of blood, plasma and serum samples collected from patients who have received a blood substitute.

### SUMMARY OF THE INVENTION

It is an object of the present invention to provide an automated method of overcoming the problem of interference during the automated analysis of whole blood, plasma and serum samples. The present invention preferably corrects for interference caused by cell free hemoglobin derivative compounds, which have a color to them and which therefore interfere with certain clinical tests and result parameters. The present invention provides an automated method to correct clinical chemistry results and hematology blood parameter results and values, e.g., mean cell hemoglobin (MCH) and mean cell hemoglobin concentration (MCHC), to account for interference error. In accordance with the present invention, automated, accurate results, free from interference error, are provided in the clinical testing and analysis of blood, plasma and serum samples collected from patients who have received a blood substitute.

Further objects and advantages afforded by the present invention will be apparent from the detailed description hereinbelow.

### DETAILED DESCRIPTION OF THE INVENTION

The present invention provides an automated method and system to account for and correct interference to hematology and clinical chemistry parameters and values. Such interference is caused by the presence of exogenous blood substitutes, e.g. cell-free hemoglobin derivatives and oxygen-carrying blood products, in blood, plasma and serum samples analyzed by automated methods and hematology systems which detect and quantify different types of hemoglobin in whole blood samples, as well as in plasma and serum samples.

Cell-free hemoglobin derivatives typically have a red color and interfere with certain clinical tests. (Z. Ma et al., 1997, Clin. Chem., 43:1732-1737). These compounds may interfere with accurate reporting of the cellular properties and blood and clinical parameters, for example, mean cell hemoglobin (MCH) and Mean Cellular Hemoglobin Concentration (MCHC). Thus, interference error is associated with the presence of cell-free hemoglobin derivatives, which carry oxygen and have a red color, in the performance of whole blood cell assays using automated hematology analyzers.

In addition to interference errors caused by hemolysis and lipemia, errors in reported results of whole blood and serum analyses can be introduced by the following: icterus, cold agglutinins, high platelet numbers, high white blood cell (WBC) counts and some medications. Generally, none of these interferences are accounted for in the automated calculation provided by the present invention. Moreover, as new blood substitutes emerge, distinct interference testing for each new substitute may be necessary.

In general, when manual calculation of the interference results occurs, there is always a greater opportunity for error compared with automated calculation and reporting. In addition, manual correction of interference due to blood substitutes added to blood samples is labor-intensive and often ignored or overlooked. The automated interference correction method described herein eliminates the chance of manual error and allows automatic reporting of accurate results in a more efficient manner.

The present invention provides the automated correction of interference error due to the use of exogenously added red, heme-colored, oxygen-carrying blood substitutes in blood samples that undergo hematology analysis. In addition, the present invention is applicable to correction of blood and clinical parameter values in whole blood, plasma and/or serum samples undergoing automated hematology analysis.

Since the blood substitutes are generally distributed in the plasma or serum fraction of the blood, blood samples containing such blood substitutes and undergoing hematology analysis appear hemolyzed. The red color in patient plasma or serum samples containing a blood substitute is due to the hemoglobin that is present in the substitute, e.g., purified hemoglobin, or derivatives thereof. However, the blood sample containing the blood substitute does not contain any of the other red blood cell interferents that are otherwise normally also present in a plasma or serum sample in which the color is due to hemolysis of endogenous red blood cells.

In view of this, interference correction should be applied for each sample containing an exogenous blood substitute only for those clinical methods which have interference from heme-color alone. The present automated correction method advantageously and specifically allows such correction to those samples requiring it by using the plasma hemoglobin value (i.e., HGB Delta, as described below) automatically generated by the automated analyzer, such as ADVIA 120®, and an appropriate correction algorithm to attain the correct value for the desired blood parameter.

According to the present invention, in addition to correcting errors in blood chemistries from patients who have been transfused with soluble heme-colored blood substitutes, the same algorithms can be used to correct for the otherwise deleterious effects of in vivo hemolysis (or in-collection-tube hemolysis in special cases where the chemistries and blood counts are performed on blood from the same collection tube).

In a preferred embodiment, automated hematology analyzers produced by and commercially available from Bayer Corporation, the assignee hereof, have been found to be able to directly determine and measure the concentration of exogenous, i.e., extracellular, hemoglobin in a sample. Suitable instruments for carrying out the analyses of the present invention possess two analytic channels which measure the concentration of hemoglobin in a blood sample. Specifically, and by way of example, the Bayer H*™ series of hematology analyzer instruments and the Bayer ADVIA® series of hematology analyzer instrument systems (e.g., ADVIA 120®) have the capability of performing quantitative analysis on the total hemoglobin content of blood and of distinguishing the hemoglobin component derived from red blood cells from that derived from the plasma.

More particularly, the Bayer hematology analyzers, are able to determine separately and independently the cellular HGB (reported as “Calculated HGB”), as well as total hemoglobin (reported as “HGB”) in a whole blood sample. These hematology analyzers can simultaneously detect cellular hemoglobin and non-cellular hemoglobin, i.e., exogenously added hemoglobin, in a whole blood sample, and thus, can report the separate values of these measurements.

Patent application U.S. Ser. No. 60/210,625, filed Jun. 9, 2000, newly describes automated methods for determining and measuring exogenous hemoglobin in a whole blood sample in a reliable and reproducible way. The methods described therein provide the ability to repeatedly or periodically monitor, during a course or regimen of a patient's treatment, hemoglobin, or a hemoglobin product, derivative or substitute, such as a cell-free oxygen-carrying hemoglobin substitute or derivative, that has been added to the blood, plasma, and/or serum of a patient. Also described are methods for monitoring, determining and/or quantifying a hemoglobin product, or a substance containing the product (e.g., blood, plasma, serum, a physiologically acceptable solution or composition, and the like) after transfusing a patient with such a hemoglobin product, derivative or substitute, such as a cell-free, oxygen-carrying hemoglobin derivative. The disclosure further provides a system to differentiate and accurately measure the contribution of an added or exogenous hemoglobin product or blood substitute, e.g., PEG-HGB or purified hemoglobin, separately and distinctly from the contribution of cellular hemoglobin, which derives from a patient's red blood cells.

The automated analytical method and system as described calculate a specific concentration of the extracellular hemoglobin, also called plasma hemoglobin, in a blood sample from a patient who has been transfused with a hemoglobin product, or in a sample which contains extracellular hemoglobin to be detected, to allow the detection and monitoring of an extracellular hemoglobin component, even in the presence of a cellular hemoglobin component derived from the red blood cells in a given sample.

The monitoring of patient progress in patients who have received exogenous hemoglobin, e.g., PEG-HGB or purified hemoglobin, via transfusions, for example, is not possible with other commercially available analyzers and other methods, because these analyzers are not able to distinguish between the hemoglobin contributed by the exogenously provided hemoglobin substitute and the hemoglobin contributed by the red blood cells in a whole blood sample. However, the automated analyzers as described herein can calculate a specific concentration of the extracellular hemoglobin in a whole blood sample from a patient into whom a hemoglobin product has been transfused, thereby allowing the detection and monitoring of exogenous hemoglobin separately from the cellular hemoglobin component derived from the red blood cells in a given sample. Thus, such analyzers provide both the cellular and total hemoglobin values for a blood sample containing an added hemoglobin product.

In a preferred embodiment of the present invention, the automated method as described is particularly applicable to and advantageous for automated methods and hematology systems designed to specifically and accurately detect, quantify and monitor different types of exogenous hemoglobin substitutes in a blood, plasma, or serum sample, preferably a whole blood sample, undergoing analysis. The present method is particularly applicable to removing interference error caused by a cell-free hemoglobin derivative or a synthetic form of hemoglobin, which has been transfused into a patient requiring added HGB, or otherwise added to a blood sample (i.e., exogenous hemoglobin).

As a consequence of interference, assays of patients' blood samples for numerous blood chemistries and hematology parameters, including, but not limited to, for example, albumin, alkaline phosphatase (ALP), alanine transaminase (ALT; formerly SGPT), amylase, aspartate transaminase (AST), urea, calcium, creatinine kinase (CK), bicarbonate, creatinine, creatinine phosphokinase, muscle/brain (CKMB), total bilirubin, gamma glutamyl transferase (GGT), glucose, lactate dehydrogenase (LDH), magnesium, phosphate, lipase, mean cell hemoglobin (MHC), mean cell hemoglobin concentration (MCHC), and preferably, albumin, ALP, amylase, calcium, bicarbonate, GGT, LDH, MCH, MCHC and total bilirubin, which are frequently affected by the presence of exogenous hemoglobin and other heme-colored oxygen-carrying blood substitute products, may not be completely accurate or correct. Accordingly, by application of the presently-described automated method, the parameter results from the automated analysis of blood samples containing blood substitutes can be corrected to accurately account for interference error, so as to achieve valid and reliable values for such blood chemistry and hematology parameter results.

The method of the present invention describes a means to provide accurate, interference-free, automated results for blood samples collected from patients who have received a blood substitute. This method is more convenient than manual calculations and is not currently available. Using the method of the present invention, automated clinical analysis of patients' blood samples and the monitoring of progress of patients who have received a blood substitute can occur along with simultaneous automated correction of blood chemistry and hematology values, for example, MCH and MCHC values, that are clinically determined and reported for these samples.

In accordance with the practice of the present invention, in addition to the standard tube-label, blood collection tubes from patients who have received a blood substitute have one or more additional, special descriptive stickers, adhesives, labels, and the like, e.g., a piece of tape, or an applied sticker paper, attached or affixed thereto. Such stickers, adhesives, or labels, which may be color-coded, bar-coded, and/or contain other easily readable markings, alert laboratory personnel that samples contain, or do not contain, a blood substitute. Each sample container or tube is assigned an identification, for example, a sample identification number assigned to the sample (Sid#), or a sequence number (Seq#), related to the position of the sample in relation to other samples in the analyzer. Work orders for each sample are typically generated by the Data Manager or by the laboratory information system (LIS).

In further accordance with this invention, test selectivity is generated by one or more specific control characters on a standard tube label, e.g., as part of a bar code, for hematology and/or clinical laboratory samples. For example, a specific character, marking, code, and the like, is added to the bar code label to signal the application of a correcting formula or algorithm to correct for hemoglobin interference, as described below. The character, marking, or code, and the like, can be a number, a letter, a symbol, or a series or combination thereof, as long as it specifically signals the appropriate automatic interference correction to be made to the results of the sample containing a hemoglobin substitute and undergoing assay in the tube. In the method of the present invention, each blood sample tube which holds a sample that contains a blood substitute is assigned a distinctive character or code, unlike any other, to identify patient samples containing a blood substitute. It is not essential that the character or code be of a specific or defined type, so long as the character or code clearly and adequately identifies a given blood sample tube as one that contains a blood substitute. For example, as mentioned above, the distinctive character or code could appear as part of a bar code label affixed to the blood sample tube.

The bar code label thus signals, i.e., triggers, the application of the automated correction formula (i.e., algorithm) in the software of the automated analyzer according to the present invention for obtaining corrected clinical/laboratory values for blood chemistries, including, but not limited to, MCH and MCHC, for example. Thus, following such a signal or trigger, the correction for interference is newly and automatically performed/determined by the automated analyzer using the plasma hemoglobin value (or HGB Delta) that is automatically provided by the analyzer. The analyzer, in turn, automatically applies a suitable algorithm or formula comprising the correction factor which is appropriate for any separate blood samples from the same patient drawn at the same time for a particular blood chemistry value already stored or scheduled in the laboratory information system (LIS), e.g, a bilirubin chemistry result (see Example 4), an albumin chemistry result, an ALP chemistry result, an LDH chemistry result, an MCH result, and/or an MCHC result, to correct for erroneously elevated values due to the interference of the exogenous hemoglobin substitute.

As will be appreciated by the skilled practitioner in the art, correction of a particular test result is generally applied in the form of an algorithm comprising a constant that is added to, subtracted from and/or multiplied by a reported result or parameter from the automated analyzer on which the blood chemistry analysis is performed. For example, to obtain a corrected test chemistry result, the reported value for a particular blood chemistry parameter is used; the automatically determined plasma hemoglobin (i.e., HGB Delta) value is used, and the correction factor is used by the automated analyzer to yield a value that removes the interference of exogenous hemoglobin from the final chemistry result, e.g., total bilirubin. (see Example 4).

Accordingly, in one of its aspects, the automated method of the present invention corrects the parameters of mean cell hemoglobin (MCH), (units: picograms/cell), and mean cell hemoglobin concentration (MCHC), (units: gm/L), values in a sample, particularly, a whole blood sample, containing a heme-colored interfering substance and comprises dividing the cellular hemoglobin concentration (units: gm/dL) by the red blood cell concentration (units: cells/mm³) to obtain a first value; multiplying the first value by a first constant, e.g., 10, to correct for differences in units of dimensions to obtain a corrected MCH value; dividing the cellular hemoglobin concentration (units: gm/dL) by the hematocrit (HCT) value, (%) to obtain a second value; and multiplying the second value by a second constant, e.g., 100, to correct for differences in units of dimensions so as to obtain a corrected mean cell hemoglobin concentration.

The present method is particularly advantageous because a number of cell-free hemoglobin substitutes and derivatives have been developed for use instead of whole blood, especially in trauma cases. These hemoglobin substitutes and derivatives can cause interference with the reported values for blood and blood chemistry parameters obtained from automated analyzers. Thus, the method according to the present invention provides an advantageous and convenient way to correct for interference error associated with hematology and clinical chemistry values reported via automated analyzers that determine, measure and monitor levels of such cell-free hemoglobin and oxygen-carrying products are added exogenously to blood, and introduced (e.g., transfused) into patients as a substitute for whole blood.

It will be appreciated that the method of the present invention applies to the analysis of blood samples, preferably whole blood samples, from patients who have received cell-free red blood cell substitutes, i.e., who have added hemoglobin, or heme-colored, oxygen-carrying blood substitute products in their blood, for a variety of medical reasons. It will be further appreciated that there are a number of cell-free, hemoglobin-based red blood cell substitutes which can be added to blood, or used as blood substitutes, to treat patients requiring such red blood cell or oxygen-carrying blood substitutes, for various therapies and treatment conditions, such as transfusion, restoration of blood volume, treatment of acute blood loss, surgery, shock (e.g., hemorrhagic shock), or tumor oxygenation, for example.

Nonlimiting examples of cell-free, hemoglobin-based red blood cell substitutes, or oxygen-carrying substitutes, that can be determined, measured, and/or monitored in whole blood samples in accordance with the present methods include cross-linked, particularly chemically cross-linked, human hemoglobin products (e.g., D. J. Nelson, 1998, “HemAssist: Development and Clinical Profile”, In: Red Blood Cell Substitutes, 1998, (Eds.) A. S. Rudolph, R. Rabinovici, and G. Z. Feuerstein, Dekker, New York, N.Y., pp. 353-400; J. Adamson et al., 1998, Ibid., pp. 335-351; and T. M. S. Chang, 1998, Ibid., pp. 465-473); recombinant hemoglobin products, particularly recombinant human hemoglobin (e.g., J. H. Siegel et al., 1998, Ibid., pp.119-164 and J. W. Freytag and D. Templeton, 1998, Ibid., pp. 325-222); purified, preferably, ultrapurified human hemoglobin products; and animal-based oxygen-carrying products, for example, bovine hemoglobin-based oxygen carrier products, e.g., Hemopure® (Cambridge, Mass.), involving purified animal (e.g., bovine) hemoglobin, or recombinant animal (e.g., bovine) hemoglobin. (W. R. Light et al., 1998, Ibid., pp. 421-436, and T. Standl et al., 1998, Br. J. Anaesth., 80(2):189-194). The use of automated hematology analyzers in the methods according to the present invention provides further advantages, which are described herein and demonstrated by the Examples as set forth below.

Patent application U.S. Ser. No. 60/210,625, filed Jun. 9, 2000, describes a method by which a commercially available automated hematology analyzer is able to detect simultaneously the intracellular (or cellular) hemoglobin (i.e., “Calculated HGB”) and extracellular HGB (i.e., “HGB Delta or HGBΔ”) in a whole blood sample. Thus, hemoglobin substitutes added to blood can be monitored by automatically determining the amount of added hemoglobin substitute independently of the hemoglobin contributed by the red blood cell component of blood. (See Example 1). For normal and abnormal unlysed blood samples, with a properly calibrated automated system, HGB Delta equals zero.

Hematology analyzers suitable for performing the simultaneous detection of intracellular and extracellular hemoglobin, e.g., Bayer ADVIA 120® and the Bayer H*™ System series of hematology analyzers, are able to directly measure the concentration of exogenous extracellular hemoglobin because these instruments possess two analytic or detection channels, each of which measures a different type of hemoglobin concentration in a whole blood sample.

In such instruments, one of the analytic or detection channels is the Hemoglobin (HGB) channel which measures the concentration of total hemoglobin in the sample by means of hemolysis and extraction of the hemes from their biological complex with globin, forming a ligated ferric heme species which is captured in a surfactant micelle and is measured spectrophotometrically (See, for example, U.S. Pat. No. 5,858,794 to M. Malin; M. Malin et al., 1992, Anal. Chim. Acta, 262:67-77; and M. Malin et al., 1989, Am. J. Clin. Path., 92:286-294). The second analytic or detection channel in such instruments is the Red Blood Cell (RBC) channel which measures the red blood cell concentration and the mean cell volume (MCV) and mean cellular hemoglobin concentration (MCHC) of approximately 10,000 individual erythrocytes as they pass through two light scattering detectors.

The presence and design of hematology analyzers having both an HGB channel and an RBC channel, in conjunction with two light scattering detectors which detect the light scattered on a cell-by-cell basis as a blood sample containing RBCs passes through the RBC optical channel, allow a difference between intracellular hemoglobin and extracellular hemoglobin to be determined and calculated, thereby providing the automatic determination of the exogenous hemoglobin component in a blood sample. For a description of the optical mechanisms of suitable automated analyzers that are capable of performing the method of the present invention, see Kim and Ornstein, 1983, Cytometry, 3:419-427; U.S. Pat. No. 4,412,004 to Ornstein and Kim; Tycko et al., 1985, Appl. Optics, 24:1355-1365; U.S. Pat. No. 4,735,504 to Tycko; and Mohandas et al., 1986, Blood, 68:506-513.

The method of measuring and determining the intracellular versus extracellular, or exogenously added, HGB concentration in a whole blood sample, as well as the total HGB concentration, is capable of being used and performed on any of the commercially available Bayer H*™ System or ADVIA 120®) hematology analyzer instruments. However, it will be understood by those having skill in the pertinent art that other hematology instruments having a two channel system of measuring HGB concentration in the blood can be designed to automatically determine HGB Delta value to be suitable for performing the interference correction method as described herein. Further, a series or combination of hematology and chemistry analyzers which are designed and/or programmed to operate on the basis of a two channel hemoglobin analysis and correction system are also contemplated for use.

The present automated method of correcting for inaccurate blood parameter values in blood samples containing one or more interference substances, such as an exogenously added hemoglobin component or derivative, encompasses blood sample values obtained from samples subjected to automated hematology analysis. In a particular aspect in accordance with the present invention, the MCH and MCHC parameter values are corrected on the Bayer ADVIA 120® hematology analyzer by the automated correction method, as follows:

MCH (corrected), [units: picograms/cell]=

$$M C H ( corrected ) , [ units : picograms \text{/} cell ] = \frac{R B C H G B [ units : gm \text{/} dL ]}{RBC concentration [ units : cells \text{/} {mm}^{3} ]} ( \times 10 )$$

$$and$$

$$MCHC ( corrected ) , [ units : gm \text{/} dl ] = \frac{R B C H G B [ units : gm \text{/} dL ]}{H C T ( \% )} ( \times 100 )$$

In the above steps of the method, RBC HGB is cellular HGB, RBC is red blood cell concentration (cells/mm³) and HCT is hematocrit, the percent of the blood volume occupied by red blood cells. (×10) and (×100) are numerical constants to correct for differences in dimensions. In accordance with the present invention, plasma hemoglobin (reported as HGB Delta) is not needed to correct the MCH and MCHC values, because cellular hemoglobin is a reported parameter in the automated hematology method described herein and the present method provides a simpler, automated correction of these values.

In contrast to the present invention, non-automated correction of MCH and MCHC values requires three manual steps. In addition, the plasma hemoglobin value must be manually and separately determined and then used for manual calculation (See, Examples 2 and 3). The manual calculation of MCH and MCHC involving three steps is exemplified as follows:

(1) RBC HGB=Total HGB−Plasma HGB, [units: gm/dL];

$$\begin{matrix}R B C H G B = Total HGB - Plasma HGB , [ units : gm \text{/} dL ] ; & ( 1 ) \\ MCH , ( corrected ) , [ units : picograms \text{/} cell ] = \frac{RBC HGB}{RBC count , [ count units : cells \text{/} {mm}^{3} ]} ( \times 10 ) ; \\ and & ( 2 ) \\ MCHC ( corrected ) , [ units : gm \text{/} dl ] = \frac{R B C H G B , [ units : gm \text{/} dL ]}{H C T [ \% ]} ( \times 100 ) . & ( 3 )\end{matrix}$$

In experiments employing PEG-HGB as a specific, yet nonlimiting, example of an exogenous hemoglobin substitute in a blood sample, the automated interference detection and correction method of this invention was demonstrated using blood samples containing exogenously added PEG-hemoglobin. The examples herein demonstrate the manual calculations required to correct for interference in a patient's blood sample. Such manual calculations are labor-intensive and inefficient compared with the simpler, automated correction method provided by the present invention.

### EXAMPLES

The following examples as set forth herein are meant to illustrate and exemplify the various aspects of carrying out the present invention and are not intended to limit the invention in any way.

### Example 1

### Automated Hematology Analysis. ADVIA 120® Hematology Analyzer (Bayer Corporation

The use of automated hematology analyzer analysis, such as the ADVIA 120® analyzer, (Bayer Corporation) allowed the detection and measurement of extracellular (or non cell-derived) hemoglobin, e.g., PEG-HGB, that was added to anticoagulated whole blood samples.

The added HGB component in a blood sample was obtained by determining the difference between the total HGB (computed from the calorimetric absorbance in the hemoglobin channel of the hematology analyzer) and the calculated cellular HGB (derived from the red blood cell (RBC) cytogram in the red cell channel of the hematology analyzer), which was calculated by the formula: (RBC×MCV×CHCM/1000), where MCV is mean cell volume and CHCM is Cellular Hemoglobin Concentration Mean, which measures the same cellular property as MCHC or Mean Cellular Hemoglobin Concentration in unlysed blood. The CHCM value is obtained from the Red Blood Cell channel of the hematology analyzer, such as the ADVIA 120® hematology system.

In particular, CHCM was obtained from light scattering measurements according to Mie Theory (see Tycko et al., 1985, Appl. Optics, 24:1355-1365, and U.S. Pat. No. 4,735,504 to Tycko). In contrast, MCHC was obtained by dividing total HGB by the product (MCV×RBC). Practically speaking, MCHC is not exactly equal to CHCM in normal samples, but these values preferably agree closely. For example, the MCHC value associated with the added HGB component is preferably within a range of about 0-5 g/dL of blood, more preferably between about 0-2 g/dL of blood, of the CHCM value. The difference between total hemoglobin and intracellular hemoglobin is termed “HGB Delta” (“HGBΔ”) and represents the concentration of exogenously added hemoglobin, e.g., PEG-HGB, in a blood sample.

An explanation related to the above-mentioned lack of complete equality between the MCHC and CHCM values is as follows. After a typical meal, for example, it is not uncommon for the blood plasma to develop a small degree of lipemia (i.e., a suspension of small submicroscopic and microscopic particles of lipids, called chylomicrons). The presence of the particles causes a minor amount of light scattering, thereby diminishing the amount of light transmitted through a solution of hemoglobin in a hemoglobinometer. Consequently, the solution appears to contain slightly more hemoglobin than it actually does. The cell by cell measurements of hemoglobin concentration, performed by the Bayer ADVIA 120® hematology analyzer, are free of this error. The ADVIA 120® is calibrated such that if ΔHGB is greater than 1.9 gm/dL, a sample is flagged as abnormal; i.e., a degree of lipemia in excess of this amount is considered abnormal. Also, if part of the blood sample has hemolysed, either in vivo in the patient or in the collection tube, a ΔHGB value is also produced. The two HGB measurements performed by the ADVIA 120® analyzer alert the physician or clinician to the existence of any abnormal lipemia or hemolysis in a patient sample.

The Bayer ADVIA 120® hematology analyzer calculates the difference (“HGB Delta”, or “HGBΔ”) between the total and intracellular HGB concentrations (all HGB concentrations are in grams per deciliter, g/dL, of whole blood), as follows:

HGBΔ, g/dL=Total HGB, g/dL_{HGB Channel}−Intracellular HGB, g/dL_{Red Cell Channel}.

In the above equation, HGBΔ represents the concentration of the extracellular HGB in the blood sample. Under ordinary conditions, delta HGB is equal to zero (0).

Thus, total hemoglobin was measured and monitored using the HGB channel of the hematology instrument, while the RBC channel detected only the intracellular HGB contained within the red blood cells of a blood sample. These two measurements were subtracted to yield the HGB Delta, which represents extracellular HGB.

The total and intracellular hemoglobin concentration values were used to calculate the difference between total and intracellular hemoglobin concentrations so as to arrive at the value for the extracellular hemoglobin (i.e., plasma hemoglobin) concentration in the blood sample, a value which was calculated automatically by the hematology analyzer. The red cell channel of the hematology analyzer measured the hemoglobin concentration in whole blood as follows:

[HGB]_{Blood, Red Cell Channel/Intracellular} (g/dL)=[CHCM (g/dL)×RBC Count (cells/mm³)×MCV (femtoliters/cell)/1000].

The HGB channel measured the total hemoglobin concentration, i.e.,

[HGB]_{intracellular}+[HGB]_{Extracellular}

The automated hematology analyzer, e.g., ADVIA 120® (Bayer Corporation), calculated the difference between the Total HGB concentration and Intracellular HGB concentration to yield the HGB Delta, which corresponds to the extracellular or exogenous HGB concentration.

Materials and Methods

PEG HGB (Enzon, Inc., Piscataway, N.J.) was received frozen and stored frozen in the freezer. The frozen bag was thawed prior to using and was transferred into five 50 ml polypropylene test tubes. Three tubes were refrozen for later use; the other two tubes were stored in the refrigerator. An appropriate aliquot for each experiment was decanted into a test tube and was allowed to equilibrate to room temperature prior to use.

The experiments described herein were performed on a calibrated Bayer Corporation ADVIA 120®) automated hematology instrument. The hemoglobin channel on this instrument utilized a cyanide-containing HGB reagent, such as that described in U.S. Pat. No. 5,858,794 to M. Malin, and a Red Blood Cell Diluent (RBC Diluent), as described in U.S. Pat. No. 5,817,519 to D. Zelmanovic et al. and U.S. Ser. No. 08/884,595, filed Jun. 27, 1997 to D. Zelmanovic et al.).

To calibrate the ADVIA 120® hematology system, the calibrator material (ADVIA 120® Setpoint™ calibrator) was aspirated ten times, and the mean HGB value was determined. The system calibrator factor was then set such that the mean calibrator value corresponded to the label value for HGB (g/dL) on the calibrator. To estimate the precision of the HGB channel, a freshly drawn whole blood sample was aspirated twenty times and the mean and standard deviation (SD) were calculated. Acceptable precision was as follows: SD<0.11 g/dL.

Blood samples obtained from normal volunteers were anticoagulated, preferably using K₃EDTA (≅12.15 mg/tube).

Most of the experiments described in the examples herein were performed at HGB concentration levels of approximately 6 g/dL, since PEG-HGB (Enzon, Inc.) is reported to contain 6 g/dL of bovine hemoglobin. The recovered HGB value was 5.4±2 g/dL, which correlated well with the nominal value 6 g/dL of blood. In addition, in all of the examples utilizing a Bayer Corporation hematology analyzer, the hemoglobin precision of the instrument was checked prior to calibration by twice aspirating PEG-HGB prior to each experiment.

### Example 2

The detection of interference and the application of calculations (both manual and automated according to the present invention) to correct the blood analysis results for interference to MCH and MCHC values are demonstrated in this example. Linearity pools of PEG-HGB (Enzon, Inc.) and diluted whole blood (diluted with plasma) were made.

The original assay of PEG-HGB was as follows: Total HGB: 5.4 g/dL; HGB Delta: 5.4 g/dL. An aliquot of a mixture containing 20% PEG-HGB and 80% diluted whole blood was assayed on the Bayer ADVIA 120® hematology instrument. Tables 1A and 1B show a comparison of the blood parameters obtained from a control, diluted whole blood sample (Table 1A) and those obtained from the sample containing a mixture of 20% PEG-HGB and 80% diluted whole blood. Table 1B presents the corrected values for MCH and MCHC obtained according to the present automated method.

TABLE 1A
Diluted Whole Blood

| Total HGB | 6.2 | g/dL |
|---|---|---|
| Cell HGB | 6.0 | g/dL |
| HGB Delta | 0.1 | g/dL |
| RBC | 2.16 × 10⁶ cells/μL | |
| HCT | 18.2% | |
| MCH | 28.6 | pg |
| MCHC | 33.8 | g/dL |
| CHCM | 33.1 | g/dL |

TABLE 1B
20% PEG + 80% Diluted Whole Blood

Corrected Value

| | | |
|---|---|---|
| Total HGB | 6.0 | g/dL |
| Cell HGB | 4.9 | g/dL |
| HGB Delta | 1.1 | g/dL |

| | |
|---|---|
| RBC | 1.75 × 10⁶ |
| | cells/μL |
| HCT | 14.8% |

| | | | | |
|---|---|---|---|---|
| MCH | 34.4 | pg | 28 | pg |
| MCHC | 40.6 | g/dL | 33.1 | g/dL |
| CHCM | 33.0 | g/dL | | |

Manual, unautomated corrections for MCH and MCHC in the aliquot sample were performed as set forth in the following three steps:

Step 1) Total HGB−Plasma HGB (HGB Delta)=Red Blood Cell HGB 6.0−1.1 gm/L=4.9 gm/dL

Step 2) Red Blood Cell HGB/RBC (×10)=MCH (Corrected) 4.9 g/dL/1.75 cells/mm³ (×10)=28.0 picograms/cell

Step 3) Red Blood Cell HGB/HCT (×100)=MCHC (Corrected) 4.9 g/dL/14.8 (×100)=33.1 gm/dL

Using the automated ADVIA 120® hematology analyzer, the automated method for correction was employed according to the present invention. In the automated method, it was not necessary to perform a calculation for plasma HGB, because HGB is a reported parameter in the automated system. The calculations employed in the automated correction method were as follows:

Step 1) Cell HGB/RBC concentration (×10)=MCH (Corrected) 4.9/1.75 (×10)=28.0 picograms/cell

Step 2) Cell HGB HCT (×100)=MCHC (Corrected) 4.9/14.8(×100)=33.1 gm/dL

As calculated by the automated two-step method, the corrected MCH and MCHC values recovered the original, diluted whole blood sample results.

### Example 3

In another experiment similar to that described in Example 2, an equal volume of PEG-HGB was added to a whole blood sample. Manual and automated correction of the MCH and MCHC values are provided beneath Tables 2A and 2B. Tables 2A and 2B show a comparison of the blood parameters obtained from a control, diluted whole blood sample (Table 2A) and those obtained from the sample containing a mixture of 50% PEG-HGB and 50% diluted whole blood. Table 2B presents the corrected values for MCH and MCHC obtained according to the present automated method.

TABLE 2A
Diluted Whole Blood

| Total HGB | 13.4 | g/dL |
|---|---|---|
| Cell HGB | 13.1 | g/dL |
| HGB Delta | 0.3 | g/dL |
| RBC | 5.06 × 10⁶ cells/μL | |
| HCT | 39.6% | |
| MCH | 26.4 | pg |
| MCHC | 33.8 | g/dL |
| CHCM | 33.2 | g/dL |

TABLE 2B
50% PEG + 50% Diluted Whole Blood

Corrected Value

| | | |
|---|---|---|
| Total HGB | 9.5 | g/dL |
| Cell HGB | 6.7 | g/dL |
| HGB Delta | 2.8 | g/dL |

| | |
|---|---|
| RBC | 2.56 × 10⁶ |
| | cells/μL |
| HCT | 20.0% |

| | | | | |
|---|---|---|---|---|
| MCH | 37.1 | pg | 26.2 | pg |
| MCHC | 47.6 | g/dL | 33.5 | g/dL |
| CHCM | 33.5 | g/dL | | |

Manual correction of the MCH and MCHC values required three steps, as follows:

Step 1) Total HGB−Plasma HGB (HGB Delta)=RBC HGB 9.5−2.8=6.7 gm/dL

Step 2) RBC HGB RBC concentration (×10)=MCH (Corrected) 6.7/2.56 (×10)=26.2 picograms/cell

Step 3) RBC HGB/HCT (×100)=MCHC (Corrected) 6.7/20.0 (×100)=33.5 gm/dL

By using only two steps in conjunction with the automated analysis of a blood sample on an automated hematology analyzer such as the ADVIA 120® (Bayer Corporation), the automated analyzer recovered the original whole blood values for MCH and MCHC. Thus, according to the present invention, the automated correction method involved two calculation steps as follows:

Step 1) RBC HGB RBC concentration (×10) MCH (Corrected) 6.7/2.56 (×10)=26.2 picograms/cell

and

Step 2) Cell HGB HCT (×100)=MCHC (Corrected) 6.7/20.0 (×100)=33.5 gm/dL

### Example 4

This example demonstrates a calculation that is used according to the present method to correct a bilirubin chemistry result, which was erroneously elevated due to the interference of exogenous hemoglobin in the blood sample undergoing testing. According to the present invention, the label on the blood sample tube triggers the software of the automated analyzer to correct for interference to total bilirubin and provide a corrected total bilirubin result following the automated application of the algorithm which comprises a correction factor constant to calculate the interference-corrected value for total bilirubin. In the calculation, plasma/serum hemoglobin (e.g., HGB Delta) is employed to obtain the corrected value, i.e., Corrected Result=Reported Result−(Correction Factor×Plasma/Serum Hemoglobin (g/L).

| | | |
|---|---|---|
| Reported total bilirubin value | 10.8 | mg/dL |
| Measured plasma / serum hemoglobin | 20.0 | g/L |
| Correction Factor | 0.13 | mg/dL/gL |

The automated Corrected result is as follows:

10.8 mg/dL−(0.13 mg/dL/gL×20.0 g/L)=8.2 mg/dL

In accordance with the present invention, the total bilirubin value is corrected automatically after analysis on the hematology analyzer.

In this example, one or more labels and/or designations on the tube housing the blood sample comprise(s) and transmit(s) a signal to the analyzer's computerized software that the sample undergoing analysis contains a blood substitute and therefore that interference correction is required for the bilirubin chemistry result. Accordingly, an algorithm or formula comprising the correction factor for bilirubin and the plasma/serum hemoglobin is automatically applied to any uncorrected total bilirubin value from the separate analysis of blood samples from the same patient drawn at the same time by the software of the automated hematology/LIS system to achieve the correct value for bilirubin adjusted for interference error, as exemplified herein.

The contents of all patents, patent applications, published articles, books, reference manuals and abstracts cited herein are hereby incorporated by reference in their entirety to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. An automated method for correcting mean cell hemoglobin (MCH) and mean cell hemoglobin concentration (MCHC) values in a blood, plasma, or sample containing a heme-colored interfering substance and analyzed on an automated hematology analyzer, comprising:
(a) dividing cellular hemoglobin concentration (gm/dL) by red blood cell concentration (cells/mm³);
(b) multiplying the value of (a) by a first constant to correct for differences in units of dimensions to obtain a corrected mean cell hemoglobin (MCH) value (gm/dL);
(c) dividing the cellular hemoglobin concentration by the hematocrit (HCT), (%), value; and
(d) multiplying the value of (c) by a second constant to correct for differences in units of dimensions to obtain a corrected mean cell hemoglobin concentration (MCHC) value (gm/dL).

2. The method according to claim 1, wherein the interfering substance in the blood sample is an extracellular hemoglobin product or an oxygen-carrying blood substitute.

3. The method according to claim 1, wherein the blood sample is a normal blood sample or an abnormal blood sample.

4. The method according to claim 1, wherein the sample is a plasma or serum sample.

5. The method according to claim 4, wherein the abnormal blood sample is derived from an individual having a pathological condition.

6. The method according to claim 5, wherein the pathological condition is selected from the group consisting of blood loss during surgery, blood loss during trauma, and hemorrhagic shock.

7. The method according to claim 2, wherein the extracellular hemoglobin product or the oxygen-carrying blood substitute is selected from the group consisting of recombinant human hemoglobin, cross-linked hemoglobin, polymerized, cross-linked hemoglobin, purified bovine hemoglobin and hemoglobin coupled to polyethylene glycol (PEG-HGB).

8. The method according to claim 2, wherein the cell-free extracellular hemoglobin product is hemoglobin isolated and purified from human or animal blood.

9. A system for alerting a practitioner of the need to correct mean cell hemoglobin (MCH) and mean cell hemoglobin concentration (MCHC) values in a blood sample containing an exogenous blood substitute, and for correcting said values using an automated hematology analyzer, comprising:
a) labeling a blood collection container to indicate that the blood sample contained therein contains an exogenous blood substitute; and
b) correcting automatically for mean cell hemoglobin (MCH) and mean cell hemoglobin concentration (MCHC) values based on the labeling indication of (a), wherein said correction is performed by the automated analyzer and comprises formula (1):
(1) MCH (corrected), (picograms/cell)=
$$MCH ( corrected ) , ( picograms \text{/} cell ) = \frac{Cellular hemoglobin ( gm \text{/} dL )}{Red Blood Cell concentration ( cells \text{/} {mm}^{3} )} ( \times constant to correct for units of dimensions ) ; \qquad ( 1 )$$
and formula (2):
(2) MCHC (corrected), (gm/dL)=
$$MCHC ( corrected ) , ( gm \text{/} dL ) = \frac{Cellular hemoglobin ( gm \text{/} dL )}{HCT ( \% )} ( \times constant to correct for units of dimensions ) ; \qquad ( 2 )$$
wherein the corrected mean cell hemoglobin (MCH) and mean cell hemoglobin concentration (MCHC) values recover the original whole blood values for mean cell hemoglobin (MCH) and mean cell hemoglobin concentration (MCHC) in the analyzed blood sample.

10. The system according to claim 9, wherein the exogenous blood substitute is an oxygen-carrying hemoglobin substitute selected from the group consisting of recombinant human hemoglobin, cross-linked hemoglobin, polymerized, crosslinked hemoglobin, purified bovine hemoglobin and hemoglobin coupled to polyethylene glycol (PEG-HGB).

11. The system according to claim 10, wherein the exogenous blood substitute is hemoglobin isolated and purified from human or animal blood.

12. The system according to claim 9, wherein the labeling of the blood container comprises a sticker affixed to the container, said sticker being color-coded and/or bar-coded to indicate that the blood sample contained therein comprises an exogenous blood substitute.

13. The system according to claim 12, wherein the labeling comprises a bar code.

14. The system according to claim 9, wherein the constant to correct for units of dimensions in formula 1 is 10, and the constant to correct for units of dimensions in formula 2 is 100.

15. A method for automatic correction of interference to a blood chemistry value in a blood, plasma, or serum sample analyzed on an automated hematology analyzer, said interference due to the presence of an exogenous blood substitute in the blood, plasma, or serum sample, comprising:
a) labeling a sample collection container to indicate that the sample contained therein contains the exogenous blood substitute,
wherein said label signals correction of the blood chemistry value; and
b) correcting automatically the blood chemistry value based on the labeling signal of (a), wherein the correction is performed by the automated hematology analyzer employing the plasma hemoglobin value automatically generated by the automated hematology analyzer; and wherein the corrected blood chemistry value recovers the original whole blood chemistry result for the blood chemistry value in the analyzed blood sample.

16. A method for automatic correction of interference to a blood chemistry value in a blood, plasma, or serum sample, said interference due to the presence of an exogenous blood substitute in the sample, comprising:
a) labeling a sample collection container to indicate that the blood, plasma, or serum sample contained therein contains the exogenous blood substitute, wherein said label signals correction of the blood chemistry value; and
b) correcting automatically the blood chemistry value based on the labeling signal of (a), wherein the correction is performed by the automated analyzer employing the plasma hemoglobin value automatically generated by the analyzer; wherein the corrected chemistry value is determined by subtracting from the reported chemistry result the following product: (correction factor x plasma or serum hemoglobin value scaled to the appropriate units of dimensions of the reported analytes); and further wherein the corrected blood chemistry value recovers the original blood chemistry result for the blood chemistry value in the analyzed sample.

17. The method according to claim 15 or claim 16, wherein the exogenous blood substitute is an oxygen-carrying hemoglobin substitute selected from the group consisting of recombinant human hemoglobin, cross-linked hemoglobin, polymerized, crosslinked hemoglobin, purified bovine hemoglobin and hemoglobin coupled to polyethylene glycol (PEG-HGB).

18. The method according to claim 17, wherein the exogenous blood substitute is hemoglobin isolated and purified from human or animal blood.

19. The method according to claim 15 or claim 16, wherein the labeling of the blood container comprises a sticker affixed to the container, said sticker being color-coded and/or bar-coded to indicate that the blood sample contained therein comprises an exogenous blood substitute.

20. The method according to claim 19, wherein the labeling comprises a bar code.

21. The method according to claim 15 or claim 16, wherein the blood chemistry value is selected from albumin, alkaline phosphatase (ALP), alanine transaminase (ALT), amylase, aspartate transaminase (AST), urea, calcium, creatinine kinase (CK), bicarbonate, creatinine, creatinine phosphokinase, muscle/brain (CKMB), total bilirubin, gamma glutamyl transferase (GGT), glucose, lactate dehydrogenase (LDH), magnesium, phosphate, lipase, mean cell hemoglobin (MHC) and mean cell hemoglobin concentration (MCHC).

22. The method according to claim 21, wherein the blood chemistry value is selected from albumin, alkaline phosphatase (ALP), amylase, calcium, bicarbonate, gamma glutamyl transferase (GGT), lactate dehydrogenase (LDH), mean cell hemoglobin (MCH), mean cell hemoglobin concentration (MCHC) and total bilirubin.

23. The method according to claim 15, wherein the corrected chemistry value is determined by subtracting from the reported chemistry result the following product: (correction factor×plasma or serum hemoglobin value scaled to the appropriate units of dimensions of the reported analytes).
